(19) United States
(12) Patent Application Publication
(10) Pub. No.: US 2005/0124967 A1
(43) Pub. Date: Jun. 9, 2005

(54) Method and device for delivery of high molecular weight substances

(76) Inventors: Scott A. Kaestner, Durham, NC (US); Ronald J. Pettis, Cary, NC (US); Diane E. Sutter, Cary, NC (US); John Mikszta, Durham, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/487,485

(22) PCT Filed: Dec. 28, 2001
(86) PCT No.: PCT/US01/50436

Related U.S. Application Data
(63) Continuation-in-part of application No. 09/835,243, filed on Apr. 13, 2001, now Pat. No. 6,569,143.
Application No. 09/835,243 is a continuation-in-part of application No. 09/417,671, filed on Oct. 14, 1999, now Pat. No. 6,494,865.
Application No. PCT/US01/50436 is a continuation-in-part of application No. 09/893,746, filed on Jun. 29, 2001.
Application No. 09/893,746 is a continuation-in-part of application No. 09/606,909, filed on Jun. 29, 2000.

(51) Int. Cl. A61M 31/00

(52) U.S. Cl. 604/506; 604/522

(57) ABSTRACT

A method and device for administration of a high molecular weight protein into the intradermal space.

### FIELD OF THE INVENTION

The invention constitutes a method and device for administration of a high molecular weight protein into the intradermal space.

### BACKGROUND OF THE INVENTION

The importance of efficiently and safely administering pharmaceutical substances such as diagnostic agents and drugs has long been recognized. Although an important consideration for all pharmaceutical substances, obtaining adequate bioavailability of large molecules such as proteins that have arisen out of the biotechnology industry has recently highlighted this need to obtain efficient and reproducible absorption (Cleland et al., Curr. Opin. Biotechnol. 12: 212-219, 2001). The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e. delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular, the papillary dermis has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue which has been far more easily and predictably targeted than the dermis has been. On the other hand, large molecules such as proteins are typically not well absorbed through the capillary epithelium regardless of the degree of vascularity so that one would not have expected to achieve a significant absorption advantage over subcutaneous administration by the more difficult to achieve intradermal administration even for large molecules.

One approach to administration beneath the surface to the skin and into the region of the intradermal space has been routinely used in the Mantoux tuberculin test. In this procedure, a purified protein derivative is injected at a shallow angle to the skin surface using a 27 or 30 gauge needle (Flynn et al, Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of substances.

Some groups have reported on systemic administration by what has been characterized as “intradermal” injection. In one such report, a comparison study of subcutaneous and what was described as “intradermal” injection was performed (Autret et al, Therapie 46:5-8, 1991). The pharmaceutical substance tested was calcitonin, a protein of a molecular weight of about 3600. Although it was stated that the drug was injected intradermally, the injections used a 4 mm needle pushed up to the base at an angle of 60°. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al. J. Pharm. Sci. 82:1175-1178, 1993). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as an intradermal drug delivery device (U.S. Pat. No. 5,997,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

To date, numerous therapeutic proteins and small molecular weight compounds have been delivered intradermally and used to effectively elicit a pharmacologically beneficial response. Most previous compounds (e.g. insulin, Neupogen, hGH, calcitonin) have been hormonal proteins not engineered receptors or antibodies. To date all administered proteins have exhibited several effects associated with ID administration, including more rapid onset of uptake and distribution (vs. SC) and in some case increased bioavailability. Little or no information is known about the behavior of high molecular weigh substances (e.g. >40 kD) when administered intradermally.

### SUMMARY OF THE INVENTION

The present disclosure relates to a new parenteral administration method based on directly targeting the dermal space whereby such method dramatically alters the pharmacokinetics (PK) and pharmacodynamics (PD) parameters of administered substances. By the use of direct intradermal (ID) administration means hereafter referred to as dermal-access means, for example, using microneedle-based injection and infusion systems (or other means to accurately target the intradermal space), the pharmacokinetics of many substances including drugs and diagnostic substances, especially high molecular weight proteins, can be altered when compared to traditional parental administration routes of subcutaneous and intravenous delivery. These findings are pertinent not only to microdevice-based injection means, but other delivery methods such as needleless or needle-free ballistic injection of fluids or powders into the ID space, Mantoux-type ID injection, enhanced iontophoresis through microdevices, and direct deposition of fluid, solids, or other dosing forms into the skin if such delivery means can be accurately controlled to deposit the drug dose within the intradermal space. Disclosed is a method to increase the rate of uptake for parenterally-administered drugs without necessitating IV access. One significant beneficial effect of this delivery method is providing a shorter Tₘₐₓ. (time to achieve maximum blood concentration of the drug). Potential corollary benefits include higher maximum concentrations for a given unit dose (Cₘₐₓ), higher bioavailability, more rapid uptake rates, more rapid onset of pharmacodynamics or biological effects, and reduced drug depot effects. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to subcutaneous, intramuscular or other non-IV parenteral means of drug delivery. Decreases in Tlag and Tmax, and more rapid absorption rates indicate faster onset for the therapeutic activity of drugs, while increased Cmax and bioavailability indicate that more drug is present in systemic circulation, and generally indicate the potential for significant reduction of doses without loss of therapeutic effect.

By bioavailability is meant the total amount of a given dosage that reached the blood compartment. This is generally measured as the area under the curve in a plot of concentration vs. time. By “lag time” is meant the delay between the administration of a compound and time to measurable or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a compound. However, numerical values can be determined more precisely by analysis using kinetic models (as described below) and/or other means known to those of skill in the art.

Directly targeting the dermal space as taught by the invention provides more rapid onset of effects of drugs and diagnostic substances. The inventors have found that substances can be rapidly absorbed and systemically distributed via controlled ID administration that selectively accesses the dermal vascular and lymphatic microcapillaries, thus the substances may exert their beneficial effects more rapidly than SC administration. This has special significance for drugs requiring rapid onset, such as insulin to decrease blood glucose, pain relief such as for breakthrough cancer pain, or migraine relief, or emergency rescue drugs such as adrenaline or anti-venom. Natural hormones are also released in pulsatile fashion with a rapid onset burst followed by rapid clearance. Examples include insulin that is released in response to biological stimulus, for example high glucose levels. Another example is female reproductive hormones, which are released at time intervals in pulsatile fashion. Human growth hormone is also released in normal patients in a pulsatile fashion during sleep. This benefit allows better therapy by mimicking the natural body rhythms with synthetic drug compounds. Likewise, it may better facilitate some current therapies such as blood glucose control via insulin delivery. Many current attempts at preparing “closed loop” insulin pumps are hindered by the delay period between administering the insulin and waiting for the biological effect to occur. This makes it difficult to ascertain in real-time whether sufficient insulin has been given, without overtitrating and risking hypoglycemia. The more rapid PK/PD of ID delivery eliminates much of this type of problem.

Mammalian skin contains two layers, as discussed above, specifically, the epidermis and dermis. The epidermis is made up of five layers, the stratum corneum, the stratum lucidum, the stratum granulosum, the stratum spinosum and the stratum germinativum and the dermis is made up of two layers, the upper papillary dermis and the deeper reticular dermis. The thickness of the dermis and epidermis varies from individual to individual, and within an individual, at different locations on the body. For example, it has been reported that the epidermis varies in thickness from about 40 to about 90 μm and the dermis varies in thickness ranging from just below the epidermis to a depth of from less than 1 mm in some regions of the body to just under 2 to about 4 mm in other regions of the body depending upon the particular study report (Hwang et al., Ann Plastic Surg 46:327-331, 2001; Southwood, Plast. Reconstr. Surg 15:423-429, 1955; Rushmer et al., Science 154:343-348, 1966).

As used herein, intradermal is intended to mean administration of a substance into the dermis in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e. the papillary dermis or in the upper portion of the relatively less vascular reticular dermis such that the substance readily diffuses into the papillary dermis. It is believed that placement of a substance predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of macromolecular and/or hydrophobic substances. Placement of the substance predominately at greater depths and/or into the lower portion of the reticular dermis is believed to result in the substance being slowly absorbed in the less vascular reticular dermis or in the subcutaneous region either of which would result in reduced absorption of macromolecular and/or hydrophobic substances. The controlled delivery of a substance in this dermal space below the papillary dermis in the reticular dermis, but sufficiently above the interface between the dermis and the subcutaneous tissue, should enable an efficient (outward) migration of the substance to the (undisturbed) vascular and lymphatic microcapillary bed (in the papillary dermis), where it can be absorbed into systemic circulation via these microcapillaries without being sequestered in transit by any other cutaneous tissue compartment.

Another benefit of the invention is to achieve more rapid systemic distribution and offset of drugs or diagnostic agents. This is also pertinent for many hormones that in the body are secreted in a pulsatile fashion. Many side effects are associated with having continuous circulating levels of substances administered. A very pertinent example is female reproductive hormones that actually have the opposite effect (cause infertility) when continuously present in the blood.

Another benefit of the invention is to achieve higher bioavailabilities of drugs or diagnostic agents. This effect has been most dramatic for ID administration of high molecular weight substances, especially proteins. The direct benefit is that ID administration with enhanced bioavailability, allows equivalent biological effects while using less active agent. This results in direct economic benefit to the drug manufacturer and perhaps consumer, especially for expensive protein therapeutics and diagnostics. Likewise, higher bioavailability may allow reduced overall dosing and decrease the patient's side effects associated with higher dosing.

Another benefit of the invention is the attainment of higher maximum concentrations of drugs or diagnostic substances. The inventors have found that substances administered ID are absorbed more rapidly, with bolus administration resulting in higher initial concentrations. This is most beneficial for substances whose efficacy is related to maximal concentration. The more rapid onset allows higher Cₘₐₓ values to be reached with lesser amounts of the substance. Therefore, the dose can be reduced, providing an economic benefit, as well as a physiological benefit since lesser amounts of the drug or diagnostic agent has to be cleared by the body.

Another benefit of the invention is no change in systemic elimination rates or intrinsic clearance mechanisms of drugs or diagnostic agents. All studies to date by the applicants have maintained the same systemic elimination rate for the substances tested as via IV or SC dosing routes. This indicates this dosing route has no change in the biological mechanism for systemic clearance. This is an advantageous from a regulatory standpoint, since degradation and clearance pathways need not be reinvestigated prior to filing for FDA approval. This is also beneficial from a pharmacokinetics standpoint, since it allows predictability of dosing regimes. Some substances may be eliminated from the body more rapidly if their clearance mechanism are concentration dependent. Since ID delivery results in higher Cmax, clearance rate may be increased, although the intrinsic mechanism remains unchanged.

Another benefit of the invention is no change in pharmacodynamic mechanism or biological response mechanism. As stated above, administered drugs by the methods taught by the applicants still exert their effects by the same biological pathways that are intrinsic to other delivery means. Any pharmacodynamic changes are related only to the difference patterns of appearance, disappearance, and drug or diagnostic agent concentrations present in the biological system.

Using the methods of the present invention, pharmaceutical compounds may be administered as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

Another benefit of the invention is removal of the physical or kinetic barriers invoked when drugs passes through and becomes trapped in cutaneous tissue compartments prior to systemic absorption. Elimination of such barriers leads to an extremely broad applicability to various drug classes. Many drugs administered subcutaneously exert this depot effect—that is, the drug is slowly released from the SC space, in which it is trapped, as the rate determining step prior to systemic absorption, due to affinity for or slow diffusion through the fatty adipose tissue. This depot effect results in a lower Cₘₐₓ and longer Tₘₐₓ, compared to ID, and can result in high inter-individual variability of absorption. This effect is also pertinent for comparison to transdermal delivery methods including passive patch technology, with or without permeation enhances, iontophoretic technology, sonopheresis, or stratum corneum ablation or disruptive methods. Transdermal patch technology relies on drug partitioning through the highly impermeable stratum corneum and epidermal barriers. Few drugs except highly lipophilic compounds can breach this barrier, and those that do, often exhibit extended offset kinetics due to tissue saturation and entrappment of the drugs. Active transdermal means, while often faster than passive transfer means, are still restricted to compound classes that can be moved by charge repulsion or other electronic or electrostatic means, or carried passively through the transient pores caused by cavitation of the tissue during application of sound waves. The stratum corneum and epidermis still provide effective means for inhibiting this transport. Stratum corneum removal by thermal or laser ablation, abrasive means or otherwise, still lacks a driving force to facilitate penetration or uptake of drugs. Direct ID administration by mechanical means overcomes the kinetic barrier properties of skin, and is not limited by the pharmaceutical or physicochemical properties of the drug or its formulation excipients.

Another benefit of the invention is highly controllable dosing regimens. The applicants have determined that ID infusion studies have demonstrated dosing profiles that are highly controllable and predictable due to the rapid onset and offset kinetics of drugs or diagnostic agents delivered by this route. This allows almost absolute control over the desired dosing regimen when ID delivery is coupled with a fluid control means or other control system to regulate metering of the drug or diagnostic agent into the body. This single benefit alone is one of the principal goals of most drug or diagnostic agent delivery methods. Bolus ID substance administration as defined previously results in kinetics most similar to IV injection and is most desirable for pain relieving compounds, mealtime insulin, rescue drugs, erectile dysfunction compounds, or other drugs that require rapid onset. Also included would be combinations of substances capable of acting alone or synergistically. Extending the ID administration duration via infusion can effectively mimic SC uptake parameters, but with better predictability. This profile is particularly good for substances such as growth hormones, or analgesics. Longer duration infusion, typically at lower infusion rates can result in continuous low basal levels of drugs that is desired for anticoagulants, basal insulin, and chronic pain therapy. These kinetic profiles can be combined in multiple fashion to exhibit almost any kinetic profile desired. An example would be to pulsatile delivery of fertility hormone (LHRH) for pregnancy induction, which requires intermittent peaks every 90 minutes with total clearance between pulses. Other examples would be rapid peak onset of drugs for migraine relief, followed by lower levels for pain prophylaxis.

Another benefit of the invention is reduced degradation of drugs and diagnostic agents and/or undesirable immunogenic activity. Transdermal methods using chemical enhancers or iontophoresis, or sonophoresis or electroporation or thermal poration require that a drug pass through the viable epidermal layer, which has high metabolic and immunogenic activity. Metabolic conversion of substances in the epidermis or sequestration by immunoglobulins reduces the amount of drug available for absorption. The ID administration circumvents this problem by placing the drug directly in the dermis, thus bypassing the epidermis entirely.

Additional benefits of the invention may be achieved when high molecular weight substances are delivered intradermally. By “high molecular weigh substance” is meant a substance comprising a compound or compounds having molecular weight(s) of 40 kD or greater. Such compounds preferably have molecular weights between 40 kD and 300 kD, but may have molecular weights up to 500 kD, 1000 D or even 2000 kD or more. Benefits of the methods of the invention for administration of high molecular weight substances include:
a) Reduced dosage (drug amount) for the patient;
b) Reduced manufacturing capacity needed to obtain an equivalent number of doses; and
c) More predictable dosing across the patient population.
   It has also been found that when such high molecular weight substances are administered according to one method of the invention, an intradermal depot of the administered substance can form. Although depot formation is often undesirable, as mentioned above, in certain instances it may provide benefits for localized therapy, pharmacokinetic control, or immunological therapy. By “intradermal depot” is meant a localized concentration of the administered substance within the dermis that is released into the surrounding tissue and the bloodstream or lymphatic systems at a reduced rate compared to that obtained by direct IV access, more dilute solutions administered SC or ID over time, or with lower molecular weight analogs of the compound administered by the same route. Such depot formation is useful in the intradermal space because, as illustrated by the present case of Enbrel, higher blood concentrations than that obtained by SC injection may be obtained over several hours, enhancing biovailability or efficacy of the drug, or both. In the case of vaccines, such ID depot formulation may enhance the uptake of vaccine by Langerhans or other dendritic cells in the skin, and may enhance direct uptake of antigen by lymphatic vessels in the intradermal space. Such effects may lead to faster seroconversion, higher antibody titers, and stronger cellular immune responses (including cytotoxic T cell and cytokine production, and cellular proliferation) as well as lowering of doses necessary for those effects compared to subcutaneous administration. Depot formation is also useful in the treatment of some localized dermal medical conditions such as skin cancers, eczema, psoriasis, warts, or moles. It could also be important for treatment of localized infections (fungal, bacterial or otherwise), and effective for longer term localized pain relief, such as that needed for orthopedic injury or nerve blocks. Depot formation would also have potential benefit for cosmetic purposes such as administration of anti-wrinkle agents.

These and other benefits of the invention are achieved by directly targeting absorption by the papillary dermis and by controlled delivery of drugs, diagnostic agents, and other substances to the dermal space of skin. The inventors have found that by specifically targeting the intradermal space and controlling the rate and pattern of delivery, the pharmacokinetics exhibited by specific drugs can be unexpectedly improved, and can in many situations be varied with resulting clinical advantage. Such pharmacokinetics cannot be as readily obtained or controlled by other parenteral administration routes, except by IV access.

The present invention improves the clinical utility of ID delivery of drugs, diagnostic agents, and other substances to humans or animals. The methods employ dermal-access means (for example a small gauge needle, especially microneedles), to directly target the intradermal space and to deliver substances to the intradermal space as a bolus or by infusion. It has been discovered that the placement of the dermal-access means within the dermis provides for efficacious delivery and pharmacokinetic control of active substances. The dermal-access means is so designed as to prevent leakage of the substance from the skin and improve adsorption within the intradermal space. The pharmacokinetics of hormone drugs delivered according to the methods of the invention have been found to be vastly different to the pharmacokinetics of conventional SC delivery of the drug, indicating that ID administration according to the methods of the invention will provide improved clinical results. Delivery devices that place the dermal-access means at an appropriate depth in the intradermal space and control the volume and rate of fluid delivery provide accurate delivery of the substance to the desired location without leakage.

Disclosed is a method to increase the rate of uptake for parenterally-administered drugs without necessitating IV access. This effect provides a shorter Tₘₐₓ. Potential corollary benefits include higher maximum concentrations for a given unit dose (Cₘₐₓ), higher bioavailability, more rapid onset of pharmacodynamics or biological effects, and reduced drug depot effects.

It has also been found that by appropriate depth control of the dermal-access means within the intradermal space that the pharmacokinetics of hormone drugs delivered according to the methods of the invention can, if required, produce similar clinical results to that of conventional SC delivery of the drug.

The pharmacokinetic profile for individual compounds will vary according to the chemical properties of the compounds. For example, compounds that are relatively large, having a molecular weight of at least 1000 Daltons as well as larger compounds of at least 2000 Daltons, at least 4000 Daltons, at least 10,000 Daltons and larger and/or hydrophobic compounds are expected to show the most significant changes compared to traditional parenteral methods of administration, such as intramuscular, subcutaneous or subdermal injection. It is expected that small hydrophilic substances, on the whole, will exhibit similar kinetics for ID delivery compared to other methods.

### DESCRIPTION OF THE DRAWINGS

FIG. 1 shows concentration-time profiles of Enbrel® after intradermal (ID), subcutaneous (SC) and intravenous (IV) administration.

### DETAILED DESCRIPTION OF THE INVENTION

The present invention provides a method for therapeutic treatment by delivery of a drug or other substance to a human or animal subject by directly targeting the intradermal space, where the drug or substance is administered to the intradermal space through one or more dermal-access means incorporated within the device. Substances infused according to the methods of the invention have been found to exhibit pharmacokinetics superior to, and more clinically desirable than that observed for the same substance administered by SC injection.

The dermal-access means used for ID administration according to the invention is not critical as long as it penetrates the skin of a subject to the desired targeted depth within the intradermal space without passing through it. In most cases, the device will penetrate the skin and to a depth of about 0.5-2 mm. The dermal-access means may comprise conventional injection needles, catheters or microneedles of all known types, employed singularly or in multiple needle arrays. The dermal-access means may comprise needleless devices including ballistic injection devices. The terms “needle” and “needles” as used herein are intended to encompass all such needle-like structures. The term microneedles as used herein are intended to encompass structures no larger than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. Dermal-access means also include ballistic fluid injection devices, powder-jet delivery devices, piezoelectric, electromotive, electromagnetic assisted delivery devices, gas-assisted delivery devices, of which directly penetrate the skin to provide access for delivery or directly deliver substances to the targeted location within the dermal space. By varying the targeted depth of delivery of substances by the dermal-access means, pharmacokinetic and pharmacodynamic (PK/PD) behavior of the drug or substance can be tailored to the desired clinical application most appropriate for a particular patient's condition. The targeted depth of delivery of substances by the dermal-access means may be controlled manually by the practitioner, or with or without the assistance of indicator means to indicate when the desired depth is reached. Preferably however, the device has structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

IV-like pharmacokinetics is accomplished by administering drugs into the dermal compartment in intimate contact with the capillary microvasculature and lymphatic microvasculature. In should be understood that the terms microcapillaries or capillary beds refer to either vascular or lymphatic drainage pathways within the dermal area.

While not intending to be bound by any theoretical mechanism of action, it is believed that the rapid absorption observed upon administration into the dermis is achieved as a result of the rich plexuses of blood and lymphatic vessels in the dermis. However, the presence of blood and lymphatic plexuses in the dermis would not by itself be expected to produce an enhanced absorption of macromolecules. This is because capillary endothelium is normally of low permeability or impermeable to macromolecules such as proteins, polysaccharides, nucleic acid polymers, substance having polymers attached such as pegylated proteins and the like. Such macromolecules have a molecular weight of at least 1000 Daltons or of a higher molecular weight of at least, 2000 Daltons, at least 4000 Daltons, at least 10,000 Daltons or even higher. Furthermore, a relatively slow lymphatic drainage from the interstitium into the vascular compartment would also not be expected to produce a rapid increase in plasma concentration upon placement of a pharmaceutical substance into the dermis.

One possible explanation for the unexpected enhanced absorption reported herein is that upon injection of substances so that they readily reach the papillary dermis an increase in blood flow and capillary permeability results. For example, it is known that a pinprick insertion to a depth of 3 mm produces an increase in blood flow and this has been postulated to be independent of pain stimulus and due to tissue release of histamine (Arildsson et al., Microvascular Res. 59:122-130, 2000). This is consistent with the observation that an acute inflammatory response elicited in response to skin injury produces a transient increase in blood flow and capillary permeability (see Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172, 1971). At the same time, the injection into the intradermal layer would be expected to increase interstitial pressure. It is known that increasing interstitial pressure from values (beyond the “normal range”) of about −7 to about +2 mmHg distends lymphatic vessels and increases lymph flow (Skobe et al., J. Investig. Dermatol Symp. Proc. 5:14-19, 2000). Thus, the increased interstitial pressure elicited by injection into the intradermal layer is believed to elicit increased lymph flow and increased absorption of substances injected into the dermis.

By “improved pharmacokinetics” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}). By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the intradermal layer and administration into the reference site such as subcutaneous administration involve the same dose levels, i.e. the same amount and concentration of drug as well as the same carrier vehicle and the same rate of administration in terms of amount and volume per unit time. Thus, for example, administration of a given pharmaceutical substance into the dermis at a concentration such as 100 μg/ml and rate of 100 μL per minute over a period of 5 minutes would, preferably, be compared to administration of the same pharmaceutical substance into the subcutaneous space at the same concentration of 100 μg/ml and rate of 100 μL per minute over a period of 5 minutes.

The enhanced absorption profile is believed to be particularly evident for substances that are not well absorbed when injected subcutaneously such as, for example, macromolecules and/or hydrophobic substances. Macromolecules are, in general, not well absorbed subcutaneously and this may be due, not only to their size relative to the capillary pore size, it may also be due to their slow diffusion through the interstitium because of their size. It is understood that macromolecules can possess discrete domains having a hydrophobic and/or hydrophilic nature. In contrast, small molecules which are hydrophilic are generally well absorbed when administered subcutaneously and it is possible that no enhanced absorption profile would be seen upon injection into the dermis compared to absorption following subcutaneous administration. Reference to hydrophobic substances herein is intended to mean low molecular weight substances, for example substances with molecular weights less than 1000 Daltons, which have a water solubility which is low to substantially insoluble

The above-mentioned PK and PD benefits are best realized by accurate direct targeting of the dermal capillary beds. This is accomplished, for example, by using microneedle systems of less than about 250 micron outer diameter, and less than 2 mm exposed length. Such systems can be constructed using known methods of various materials including steel, silicon, ceramic, and other metals, plastic, polymers, sugars, biological and or biodegradable materials, and/or combinations thereof

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion. That is, at a greater depth a needle outlet with a greater exposed height will still seal efficiently where as an outlet with the same exposed height will not seal efficiently when placed in a shallower depth within the intradermal space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of substances to the dermal space.

It has also been found that by controlling the pressure of injection or infusion may avoid the high backpressure exerted during ID administration. By placing a constant pressure directly on the liquid interface a more constant delivery rate can be achieved, which may optimize absorption and obtain the improved pharmacokinetics. Delivery rate and volume can also be controlled to prevent the formation of wheats at the site of delivery and to prevent backpressure from pushing the dermal-access means out of the skin. The appropriate delivery rates and volumes to obtain these effects for a selected substance may be determined experimentally using only ordinary skill. Increased spacing between multiple needles allows broader fluid distrubtion and increased rates of delivery or larger fluid volumes. In addition, it has been found that ID infusion or injection often produces higher initial plasma levels of drug than conventional SC administration, particularly for drugs that are susceptible to in vivo degradation or clearance or for compounds that have an affinity to the SC adipose tissue or for macromolecules that diffuse slowly through the SC matrix. This may, in many cases, allow for smaller doses of the substance to be administered via the ID route.

The administration methods useful for carrying out the invention include both bolus and infusion delivery of drugs and other substances to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver a substance the dermal-access means is placed adjacent to the skin of a subject providing directly targeted access within the intradermal space and the substance or substances are delivered or administered into the intradermal space where they can act locally or be absorbed by the bloodstream and be distributed systematically. The dermal-access means may be connected to a reservoir containing the substance or substances to be delivered. The form of the substance or substances to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. Delivery from the reservoir into the intradermal space may occur either passively, without application of the external pressure or other driving means to the substance or substances to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the substance may be variably controlled by the pressure-generating means. As a result, the substance enters the intradermal space and is absorbed in an amount and at a rate sufficient to produce a clinically efficacious result.

As used herein, the term “clinically efficacious result” is meant a clinically useful biological response including both diagnostically and therapeutically useful responses, resulting from administration of a substance or substances. For example, diagnostic testing or prevention or treatment of a disease or condition is a clinically efficacious result. Such clinically efficacious results include diagnostic results such as the measurement of glomerular filtration pressure following injection of inulin, the diagnosis of adrenocortical function in children following injection of ACTH, the causing of the gallbladder to contract and evacuate bile upon injection of cholecystokinin and the like as well as therapeutic results, such as clinically adequate control of blood sugar levels upon injection of insulin, clinically adequate management of hormone deficiency following hormone injection such as parathyroid hormone or growth hormone, clinically adequate treatment of toxicity upon injection of an antitoxin and the like.

Therapeutic substances which can be used with the present invention include monoclonal antibodies, Peglyated antibodies, Pegylated proteins or any proteins modified with hydrophilic or hydrophobic polymers or additional functional groups, fusion proteins, single chain antibody fragments or the same with any combination of attached proteins, macromolecules, or additional functional groups thereof, anti-inflammatory agents, Recombinant soluble receptors, Thrombolytics, Tissue plasminogen activators, TNF-, and TNF-antagonists, other substances including all of the major therapeutics such as, for example anti-infectives, anti-allergy agents, antiarthritics, antiasthmatic agents, anti-inflammatory agents, proteins, anti-psoriasis agents and other macromolecules.

The pharmacokinetic analysis for Enbrel delivery was calculated using “non-compartmental methods using methods and techniques known in the art.

Having described the invention in general, the following specific but not limiting examples and reference to the accompanying Figures set forth various examples for practicing the dermal accessing, direct targeting drug administration method and examples of dermal administered drug substances providing improved PK and PD effects.

A representative example of dermal-access microdevice comprising a single needle was prepared from 34 gauge steel stock (MicroGroup, Inc., Medway, Mass.) and a single 28° bevel was ground using an 800 grit carborundum grinding wheel. Needles were cleaned by sequential sonication in acetone and distilled water, and flow-checked with distilled water. Microneedles were secured into small gauge catheter tubing (Maersk Medical) using UV-cured epoxy resin. Needle length was set using a mechanical indexing plate, with the hub of the catheter tubing acting as a depth-limiting control and was confirmed by optical microscopy. For experiments using needles of various lengths, the exposed needle lengths were adjusted to 0.5, 0.8, 1, 2 or 3 mm using the indexing plate. Connection to the fluid metering device, either pump or syringe, was via an integral Luer adapter at the catheter inlet. During injection, needles were inserted perpendicular to the skin surface, and were either held in place by gentle hand pressure for bolus delivery or held upright by medical adhesive tape for longer infusions. Devices were checked for function and fluid flow both immediately prior to and post injection. This Luer Lok single needle catheter design is hereafter designated SS1_{—}34.

Yet another dermal-access array microdevice was prepared consisting of 1″ diameter disks machined from acrylic polymer, with a low volume fluid path branching to each individual needle from a central inlet. Fluid input was via a low volume catheter line connected to a Hamilton microsyringe, and delivery rate was controlled via a syringe pump. Needles were arranged in the disk with a circular pattern of 15 mm diameter. Three-needle and six-needle arrays were constructed, with 12 and 7 mm needle-to-needle spacing, respectively. All array designs used single-bevel, 34 G stainless steel microneedles of 1 mm length. The 3-needle 12 mm spacing catheter-design is hereafter designated SS3_{—}34B, 6-needle 7 mm spacing catheter-design is hereafter designated SS6_{—}34A.

Yet another dermal-access array microdevice was prepared consisting of 11 mm diameter disks machined from acrylic polymer, with a low volume fluid path branching to each individual needle from a central inlet. Fluid input was via a low volume catheter line connected to a Hamilton microsyringe, and delivery rate was controlled via a syringe pump. Needles were arranged in the disk with a circular pattern of about 5 mm diameter. Three-needle arrays of about 4 mm spacing connected to a catheter as described above. These designs are hereafter designated SS3S_{—}34, SS3C_{—}34, and SS3S_{—}34_{—}3 for 1 mm, 2 mm, and 3 mm needle lengths respectively. Device Code number SS3_{—}34B was used for Enbrel delivery. No other microdevices were used, but many of these variations would be potentially useful.

Yet another dermal-access ID infusion device was constructed using a stainless steel 30 gauge needle bent at near the tip at a 90-degree angle such that the available length for skin penetration was 1-2 mm. The needle outlet (the tip of the needle) was at a depth of 1.7-2.0 mm in the skin when the needle was inserted and the total exposed height of the needle outlet 1.0-1.2 mm. This design is hereafter designated SSB1_{—}30.

### EXAMPLE I

Enbrel® (etanercept) is a tumor necrosis factor (TNF) antagonist with a molecular weight of approximately 150 kD that is used in the treatment of arthritis. The active agent in Enbrel®, which is customarily administered by SC injection, is an engineered fusion protein consisting of the 75 kD tumor necrosis factor receptor (TNFR) linked to the Fc portion of human IgG1. Enbrel® was tested as an exemplary substance to demonstrate the feasibility of delivering high molecular weight soluble receptor proteins and immunoglobulins into the ID space.

Twenty Yucatan mini-pigs were dosed each with TNRF:Fc administered intravenously (IV, 7 animals), subcutaneously (SC, 6 animals) and intradermally (IO, 7 animals). The nominal total administered dose was 20 mg in a 250 microliter volume. ID administration was accomplished using a 3-microneedle array. Device was a 3 needle array, approximately 12 mm needle spacing, 34 Ga, 1 mm length needles, at a volumetric infusion rate of 20 microliter/min with a total dosing duration of 12.5 minutes. SC dosing was via a standard 25 gauge syringe needle over several seconds. Blood samples were drawn periodically over the 10 days following administration (at 0.5, 1, 1.5, 2, 3, 4, 6, 8, 12, 24, 36, 48, 72, 96, 144, 192, and 240 hours) and analyzed for Enbrel® content.

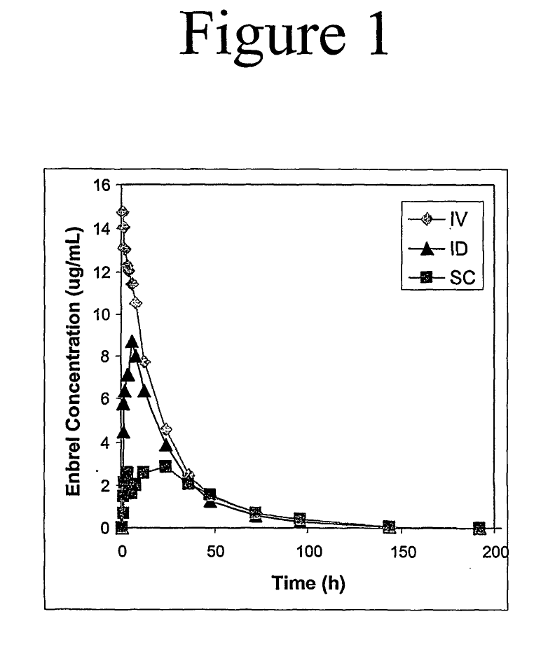

The results (FIG. 1) show that the absorption of Enbrel® following ID administration is much more rapid than via the standard SC route, with a concentration peak that occurs much earlier and is more than three times higher. In addition, absorption from the SC depot results in much higher variability in the concentration-time profile, with a secondary concentration peak occurring a significant time after the administration occurs.

A non-compartmental PK analysis was conducted on the mean concentration profiles, with the results summarized in Table 1.

TABLE 1
Summary of PK parameters from non-compartmental analysis.

| Parameter | IV | SC | ID |
|---|---|---|---|
| Tmax [hr] | 0 | 24 | 6 |
| Cmax [μg/mL] | 15.4 | 2.84 | 8.7 |
| AUClast [μg · hr/mL] | 319.3 | 158.4 | 239.4 |
| AUCinf [μg · hr/mL] | 319.4 | 158.8 | 239.5 |
| Terminal phase halflife [hr] | 17.6 | 22.8 | 19.3 |
| Bioavailability [%] | 100 | 50 | 75 |

Rapid onset of drug concentration occurs as demonstrated by the relative tₘₐₓ and Cₘₐₓ vs. SC. Relative bioavailability of the ID route is also dramatically enhanced by 50% over the current standard of SC administration. Likewise the variability between replicates is also reduced. (Note the two types of bioavailability: absolute bioavailability is calculated vs IV profiles, realtive bioavailability is calculated versus another non-IV route)

The terminal phase half-lives for all three routes of administration are within 20%, from which it can be concluded that the elimination phase is not changed by the different routes of administration.

The results show that the absolute bioavailability of TNRF:Fc is increased by 25% when administered intradermally as compared to subcutaneously. The resulting dose-sparing effect will allow administration of dosages that are 20% lower than has been possible with the standard subcutaneous injection method of the art, resulting in a large cost saving to pharmaceutical manufacturers and consumers.

The molecular weight of Enbrel® is well representative of that for natural or engineered immunoglobulins. This high molecular weight is also representative of recombinant proteins with glycosylation via large oligosacharide moieties, and representative of proteins (or other compounds) chemically modified with oligosacharides, PEG, albumin, or other moieties, to yield a desired pharmacological property such as increased circulating half-life, or “stealth” bypass of the immunological or clearance system. The postulated mechanism of uptake for ID administered drugs is partitioning into the vascular or lymphatic capillaries, with subsequent systemic distribution. The capillary cell lining of these vascular walls could have been limiting to the effective absorption of high molecular weight compound, due to limited partitioning across the cellular walls or through tight junctions. This would not have been previously detected with other proteins of lower molecular weight, but the current result does indicate the utility of the ID space for the effective absorption of high molecular weight species, which could not have been assumed. Particulate substances and suspensions have previously been demonstrated to clear from the ID space, but since most of these were vaccines or related species, no quantitative measure of the efficacy of uptake or the systemic distribution of such species could be inferred.

The high concentration of Enbrel® delivered (80 mg/mL) is also significantly greater than compounds that have been previously investigated (<15 mg/mL). Again, this unique formulation presentation could not necessarily be assumed to allow effective uptake, distribution, and biological response. Although isotonic, such a high protein concentration could upon ID injection have resulted in a localized hypertonic region resulting in altered blood or interstitial fluid flow with altered uptake kinetics or other potential failures modes including localized drug precipitation and/or localized tissue damage due to the extremely concentrated protein solution. The PK results above indicate this was not the case.

To deliver sufficient pharmacologically efficient doses of Enbrel®, the high concentrations above also had to utilize increased volumes of delivery (250 uL) than previously established. However, the increased volumes and more viscous protein solution necessitated an altered dosing regimen.

Low concentration proteins and vehicles can typically be delivered ID in swine without leakage at rates of approx. 30-60 uL/min per 1 mm, 34 Ga cannula. These rates can be readily extrapolated upward by multiplying the rate by number of microcannula for a given microdevice. In this instance a significantly slower per needle rate had to be utilized to achieve delivery without leakage. The total 250 uL volume of Enbrel® was delivered by a three needle array (12 mm needle to needle spacing) at a rate of 20 uL/min (approx. 6.7 uL/min/nedle) for a total delivery period of 12.5 minutes. Increases above this rate routinely resulted in unacceptable “wet” injections, although solution would readily flow through the cannula at these higher rates. Also, higher rates periodically resulted in localized bleeding from the injection site, probably due to localized changes in tonicity or osmolarity. Likewise, it was observed that cannula whose external walls were contaminated with Enbrel® solution would seat into the tissue, but leak upon infusion. It is postulated that the highly concentrated, viscous protein solution acts as a lubricant and prevents effective sealing around the needle cannula. To minimize tissue distension at a single site, a three-needle array was utilized to distribute the total infused volume. It is also possible that the viscoelastic properties of such formulations is highly altered from those previously examined and may be exhibiting altered microfluidic processes. Also, highly concentrated Enbrel® solutions upon short periods of standing in microcannula would rapidly precipitate or crystallize in the cannula bore, causing flow occlusion. This could be reversed by rewetting the solution externally with fluid to dissolve the occlusion.

Effective delivery of viscous, highly concentrated solutions requires:
a) Slower than expected per needle infusion rates to minimize leakage and minimize tissue damage,
b) Scrupulously clean exterior cannula walls to effect adequate tissue sealing,
c) Multiple instillation point to distribute increased volumes, and
d) Flow activation in a sufficiently hydrated environment (e.g. the interstitial space) to prevent precipitation and cannula occlusion.

Although the ID administration of Enbrel® was more efficacious than the SC standard route from a pharmacokinetic standpoint, there were significant indications of formation of an intradermal depot of drug solution with a significant lifetime. This is previously unobserved with all other proteins or drugs which are rapidly removed from the ID administration site, and exhibit peak plasma times typically of 2 hours or less. The observed tₘₐₓ for ID Enbrel® was approximately 9 hours This also correlates to the duration of the dermal wheal/bleb visually assessed in test animals. The dermal wheal usually exhibited a lifetime of 8-12 hours (measured visually by Draize edema scoring) before effective resolution began to occur. Likewise, histopathology of excised administration sites one hour after ID injection showed localized edema, and the interstitial space stained pink with a standard eosin stain indicating the presence of a proteinaceous solution in the dermal cellular bed. This staining has not previously occurred with other proteins. Also, 6-9 days post injection, a localized tissue response similar to contact dermatitis was noticed in many test animals. These sites screened positive for inflammatory cellular infiltrate into the dermis by histopathology. This is probably a species-specific immunological response of swine to a humanized protein, but does indicate that some localization of protein, or protein degradation products has occurred.

These results have not been previously demonstrated with ID administered proteins and have the following implications for administration of Enbrel®, other receptor antibodies, or high molecular weight, high viscosity solutions:
1. intradermal depot formation can be effectively utilized to:
   a) Increase a desired immunological response to a vaccine based entity;
   b) Therapeutically treat a localized cutaneous region or site such as melanoma, wart, mole, etc. with an immunological agent such as a cytotoxic substance, anti-tumor antibody, or other agent; and
   c) Generate an effective means mapping cutaneous sites for lymphatic drainage, radioimaging, or other beneficial diagnostic purpose.
2. Intradermal depot formation can be facilitated by:
   a) highly concentrated solutions,
   b) solutions of high molecular weight,
   c) bolus instillation to minimize the potential for agent effusion,
   d) high volume instillation to provide an extended lifetime, and
   e) high solution viscosity.
      Viscosity of substances to be administered can be measured by means familiar to those of skill in the art, and modified, if desired, using compounds that are not chemically or covalently bound to the therapeutically active substance, for example, PEG, PVP, PVA, sugars, in situ gel forming solutions.

Based on the above observations and results an optimized device platform for ID administration of Enbrel® or other high molecular weight compounds, or viscous solutions will exhibit the following features:
a) A slow infusion rate (<20 uL/min/cannula) to minimize device leakage due to viscosity or other formulations properties, and the potential for tissue damage;
b) An extended infusion duration (12 min-24 h) to allow effective instillation, and better match biological uptake to instillation rate and minimize depot formation;
c) Reduced solution concentrations (≦80 mg/mL) to prevent tissue site reactions, prevent leakage, prevent depots;
d) A mechanism to allow flow initiation after microcannula seating to prevent leakage or drug precipitation;
e) Multiple microcannula to spread the dose into a broader tissue space.

Based on the above observations an optimized platform for generating a dermal depot would incorporate the following features:
a) A viscosity modifier or high molecular weight additive or chemical derivative to slow absorption;
b) A reduced infusion period (<1 h) to swamp the dermal bed's ability to remove high molecular weight species; and
c) A high drug concentration to facilitate in situ depot formation.

One preferred delivery device is a mechanical infusion platform that is capable of providing
a) A slow infusion rate (<20 uL/min/cannula) to minimize device leakage due to viscosity or other formulations properties, and the potential for tissue damage;
b) An extended infusion duration (12 min-24 h) to allow effective instillation, and better match biological uptake to instillation rate and minimize depot formation;
c) Reduced solution concentrations (≦80 mg/mL)to prevent tissue site reactions, prevent leakage, prevent depots;
d) A mechanism to allow flow initiation after microcannula seating to prevent leakage or drug precipitation; and
e) Multiple microcannula to spread the dose into a broader tissue space.

Based on the above observations an optimized platform for generating a dermal depot would incorporate the following features:
a) a viscosity modifier or high molecular weight additive or chemical derivative to slow absorption;
b) a reduced infusion period (<1 h) to swamp the dermal beds ability to remove high molecular weight species; and
c) A high drug concentration to facilitate in situ depot formation
   This device will also comprise one or more intradermal microcannula (≦31 G). Suitable devices are described, inter alia, in U.S. applications Ser. No. 09/590,062, filed Jun. 8, 2000, and 09/617,355, filed Jul. 17, 2000.

Similar advantages are expected with other large molecular weight pharmaceutical compounds, including engineered or naturally occurring antibodies for therapeutic or diagnostic implications, receptor proteins, highly glycosylated low molecular weight proteins or chemically modified proteins (e.g. PEGylation, albumin conjugates, etc.)

In general, ID delivery as taught by the methods described hereto via dermal access microneedle devices provides a readily accessible and reproducible parenteral delivery route, with high bioavailability, as well as the ability to modulate plasma profiles by adjusting the device infusion parameters, since uptake is not rate-limited by biological uptake parameters.

In the previously described examples, the methods practiced by the invention demonstrate the ability to deliver a drug in vivo with greatly improved pharmaceutically relevant rates. This data indicates an improved pharmacological result for ID administration as taught by the methods described of other drugs in humans would be expected according to the methods of the invention.

All references cited in this specification are hereby incorporated by reference. The discussion of the references herein is intended merely to summarize the assertions made by their authors and no admission is made that any reference constitutes prior art relevant to patentability. Applicants reserve the right to challenge the accuracy and pertinency of the cited references.

1. A method for directly delivering a high molecular weight substance into an intradermal space within mammalian skin comprising administering the substance through at least one hollow needle having an outlet with an exposed height between 0 and 1 mm, said outlet being inserted into the skin to a depth of between 0.3 mm and 2 mm, such that delivery of the substance occurs at a depth between 0.3 mm and 2 mm.

2. The method according to claim 1 wherein the delivered substance has improved pharmacokinetics compared to pharmacokinetics after subcutaneous injection.

3. The method of claim 1 wherein the administration is through at least one small gauge hollow needle.

4. The method of claim 1 wherein the needle has an outlet with an exposed height between 0 and 1 mm.

5. The method of claim 1 wherein injecting comprises inserting the needle to a depth which delivers the substance at least about 0.3 mm below the surface to no more than about 2 mm below the surface.

6. The method of claim 1 wherein administering comprises inserting the needle into the skin to a depth of at least about 0.3 mm and no more than about 2 mm.

7. The method of claim 2 wherein the improved pharmacokinetics is increased bioavailability of the substance.

8. The method of claim 2 wherein the improved pharmacokinetics is a decrease in Tₘₐₓ.

9. The method of claim 2 wherein the improved pharmacokinetics is an increase in Cₘₐₓ.

10. The method of claim 2 wherein the improved pharmacokinetics is a decrease in T_{lag}.

11. The method of claim 2 wherein the improved pharmacokinetics is enhanced absorption rate.

12. The method of claim 1 wherein the substance is administered over a time period of not more than ten minutes.

13. The method of claim 1 wherein the substance is administered over a time period of greater than ten minutes.

14. The method of claim 1 wherein the substance is a protein.

15. The method of claim 1 wherein the substance is administered at a rate between 1 nL/min. and 200 mL/min.

16. The method of claim 1 wherein said substance is a hormone.

17. The method of claim 14 wherein said protein is a receptor protein or antibody.

18. The method of claim 17 wherein said protein is etanercept.

19. The method of claim 14 wherein said protein is a fusion protein.

20. The method of claim 1 wherein said substance has a molecular weight of at least 40,000 kD.

21. The method of claim 20 wherein said substance has a molecular weight of at least 100,000 kD.

22. The method of claim 21 wherein said substance has a molecular weight of at least 150,000 kD.

23. The method of claim 1 wherein said substance is a nucleic acid.

24. The method of claim 1 wherein said substance is hydrophobic.

25. The method of claim 1 wherein said substance is hydrophilic.

26. The method of claim 1 wherein the needle(s) are inserted substantially perpendicularly to the skin.

27. The method of claim 1 wherein administration is carried out under conditions such that an intradermal depot containing said substance is formed.

28. A method of administering a pharmaceutical substance comprising injecting or infusing the substance intradermally through one or more microneedles having a length and outlet suitable for selectively delivering the substance into the dermis to obtain absorption of the substance in the dermis.

29. The method of claim 28 wherein absorption of the substance in the dermis produces improved systemic pharmacokinetics compared to subcutaneous administration.

30. The method of claim 29 wherein the improved pharmacokinetics is increased bioavailability.

31. The method of claim 29 wherein the improved pharmacokinetics is decreased Tₘₐₓ.

32. The method of claim 29 wherein the improved pharmacokinetics is an increase in Cₘₐₓ.

33. The method of claim 29 wherein the improved pharmacokinetics is a decrease in T_{lag}.

34. The method of claim 29 wherein the improved pharmacokinetics is an enhanced absorption rate.

35. The method of claim 28 wherein the length of the microneedle is from about 0.5 mm to about 1.7 mm.

36. The method of claim 28 wherein the microneedle is 30 gauge or narrower.

37. The method of claim 28 wherein the microneedle has an outlet of from 0 to 1 mm.

38. The method of claim 28 wherein the microneedle is configured in a delivery device which positions the microneedle perpendicular to skin surface.

39. The method of claim 28 wherein the microneedle needle is contained in an array of microneedles needles.

40. The method of claim 39 wherein the array comprises 3 microneedles.

41. The method of claim 39 wherein the array comprises 6 microneedles.

42. The method of claim 28 wherein the substance has a molecular weight of at least 40 kD.

43. The method of claim 42 wherein the substance is a protein.

44. The method of claim 43 wherein said protein is a receptor protein or antibody.

45. The method of claim 44 wherein said protein is etanercept.

46. The method of claim 28 wherein administration is carried out under conditions such that an intradermal depot containing said substance is formed.

47. A microneedle for intradermal injection of a high molecular weight pharmaceutical substance, wherein the microneedle has a length and outlet selected for its suitability for specifically delivering the substance into the dermis.

48. The microneedle according to claim 47 wherein the length of the microneedle is from about 0.5 mm to about 1.7 mm.

49. The microneedle of claim 47 which is a 30 gauge or narrower.

50. The microneedle of claim 47 which has an outlet of from 0 to 1 mm.

51. The microneedle of claim 47 which is configured in a delivery device which positions the microneedle perpendicular to skin surface.

52. The microneedle of claim 47 which is in an array of microneedles needles.

53. The microneedle of claim 52 wherein the array comprises 3 microneedles.

54. The microneedle of claim 52 wherein the array comprises 6 microneedles.

55. A method for delivering a bioactive substance to a subject comprising:
contacting the skin of the subject with a device having a dermal-access means for accurately targeting the dermal space of the subject with an efficacious amount of the bioactive substance.

56. The method of claim 55 wherein the pharmacokinetics of the bioactive substance is improved relative to the pharmacokinetics of the substance when administered subcutaneously.

57. The method of claim 56 wherein the improved pharmacokinetics is an increase in bioavailability.

58. The method of claim 56 wherein the improved pharmacokinetics is a decrease in Tₘₐₓ.

59. The method of claim 56 wherein the improved pharmacokinetics comprises an increase in Cₘₐₓ of the substance compared to subcutaneous injection.

60. The method of claim 56 wherein the improved pharmacokinetics is a decrease in T_{lag}.

61. The method of claim 56 wherein the improved pharmacokinetics is an enhanced absorption rate.

62. The method of claim 55 wherein the device has a fluid driving means including a syringe, infusion pump, piezoelectric pump, electromotive pump, electromagnetic pump, or Belleville spring.

63. The method of claim 55 wherein the dermal access means comprises one or more hollow microcannula having a length of from about 0.5 to about 1.7 mm.

64. The method of claim 55 wherein said dermal access means comprises one or more hollow microcannula having an outlet with an exposed height between 0 and 1 mm.

65. The method of claim 55 wherein the substance has a molecular weight of at least 40 kD.

66. The method of claim 55 wherein the substance is or protein.

67. The method of claim 66 wherein said protein is a receptor protein or antibody.

68. The method of claim 67 wherein said protein is etanercept.

69. The method of claim 55 wherein administration is carried out under conditions such that an intradermal depot containing said substance is formed.

70. A method for delivering a high molecular weight bioactive substance to a subject comprising:
contacting the skin of a subject with a device having a dermal-access means for accurately targeting the dermal space of the subject with an efficacious amount of the bioactive substance at a rate of 1 nL/min. to 200 mL/min.

71. The method of claim 70 wherein the rapid onset pharmacokinetics of the bioactive substance is substantially improved relative to subcutaneous injection.

72. The method of claim 71 wherein the bioavailability is increased.

73. The method of claim 71 wherein the pharmokinetics is a decreased Tₘₐₓ.

74. The method of claim 71 wherein the pharmokinetics is an increased Cₘₐₓ.

75. The method of claim 71 wherein the pharmokinetics is a decreased T_{lag}.

76. The method of claim 71 wherein the pharmacokinetics is an enhanced absorption rate.

77. The method of claim 70 wherein the dermal access means has one or more hollow microcannula that inserts into the skin of said subject to a depth of from about 0.5 to about 2.0 mm.

78. The method of claim 70 wherein the dermal access means has one or more hollow microcannula having an outlet with an exposed height between 0 and 1 mm.

79. The method of claim 70 wherein said substance has a molecular weight of at least 40 kD.

80. The method of claim 70 wherein said substance is a protein.

81. The method of claim 80 wherein said protein is a receptor protein or antibody.

82. The method of claim 80 wherein said protein is etanercept.

83. The method of claim 70 wherein administration is carried out under conditions such that an intradermal depot containing said substance is formed.

84. The method of claim 80 wherein said protein is a fusion protein.

85. The method of claim 70 wherein said substance has a molecular weight of at least 100,000 kD.

86. The method of claim 85 wherein said substance is a protein.

87. The method of claim 85 wherein said substance has a molecular weight of at least 150 kD.

88. The method of claim 87 wherein said substance is a protein.
